(19) United States
(12) Patent Application Publication
(10) Pub. No.: US 2013/0273656 A1
(43) Pub. Date: Oct. 17, 2013

(54) METHOD TO INCREASE GENE TARGETING FREQUENCY

(71) Applicant: Eric Hendrickson, Minneapolis, MN (US)

(72) Inventor: Eric Hendrickson, Minneapolis, MN (US)

(73) Assignee: Regents of the University of Minnesota, St. Paul, MN (US)

(21) Appl. No.: 13/878,407

(22) PCT Filed: Oct. 7, 2011
(86) PCT No.: PCT/US11/55245

§ 371 Date: Jul. 1, 2013

Related U.S. Application Data
(60) Provisional application No. 61/391,471, filed on Oct. 8, 2010.
(60) Provisional application No. 61/438,961, filed on Feb. 2, 2011.

(51) Int. Cl. C12N 15/113

(52) U.S. Cl. CPC C12N 15/113
USPC 435/375

(57) ABSTRACT

Gene targeting is a valuable tool for basic re-searchers and gene therapists. Unfortunately, current methods utilized to target genes are inefficient because of their low targeting frequencies. Provided herein are methods and compositions by which gene targeting frequencies can be increased.

### REFERENCE TO RELATED APPLICATIONS

This application claims priority from U.S. Provisional Application Ser. Nos. 61/391,471 and 61/438,961, filed Oct. 8, 2010 and Feb. 2, 2011, respectively, each of which is incorporated herein by reference.

### STATEMENT OF GOVERNMENT RIGHTS

This invention was made with the assistance of government support under United States Grant Nos. HL079559, GM069576 and GM088351 from the National Institutes of Health. The government has certain rights in the invention.

### BACKGROUND OF THE INVENTION

Gene targeting is a valuable tool for basic researchers and gene therapists. Unfortunately, current methods utilized to target genes are inefficient because of their low targeting frequencies.

### SUMMARY OF THE INVENTION

The invention provides methods by which gene targeting frequencies can be increased. In one embodiment, the method involves the permanent inhibition of the non-homologous end joining (NHEJ) DNA double-strand break (DSB) repair pathway in cells. In an alternative embodiment, the method involves the transient inhibition of NHEJ DNA DSB repair pathway in cells. This inhibition of the NHEJ DNA DSB repair pathway can result in very high frequencies of viral, such as recombinant adeno-associated virus (rAAV)-mediated gene targeting as well as other non-viral methods of targeting (e.g., zinc finger targeting).

One embodiment provides a method for increasing gene targeting frequency (as compared to current protocols or as compared to a method without inhibition) comprising inhibiting (completely or partially) expression of a gene (RNA or protein expression) or activity of a protein of a DNA DSB repair pathway. Another embodiment provides a method for increasing targeted DNA integration (compared to current protocols or as compared to a method without inhibition) comprising inhibiting (completely or partially) expression of a gene (RNA or protein expression) or activity of a protein of a DNA DSB repair pathway. In one embodiment, the DNA DSB repair pathway is the NHEJ pathway, including the C-NHEJ and the A-NHEJ pathways. In another embodiment, the expression or activity of DNA-PK_{cs} is inhibited (for example, transiently inhibited). In another embodiment, the expression or activity of Artemis is inhibited (for example, transiently inhibited).

One embodiment provides a method to increase gene targeting frequency comprising inhibiting (completely or partially) expression (RNA or protein expression) of at least one gene of a DNA double strand break (DSB) repair pathway or by inhibiting (completely or partially) activity of at least one protein of a DNA DSB repair pathway so as to provide increased gene targeting frequency of DNA (e.g., exogenous) as compared to a cell in which expression and/or activity has not been inhibited.

Another embodiment provides a method to reduce stable random (by random it is meant that non-target DNA integrates at any location or target DNA integrates at an unintended location) DNA (e.g., exogenous) integration comprising inhibiting (completely or partially) expression (RNA or protein expression) of at least one gene of a DNA DSB repair pathway or by inhibiting (completely or partially) activity of at least one protein of a DNA DSB repair pathway so as to provide decreased exogenous DNA integration as compared to a cell in which expression and/or activity has not been inhibited, provided the exogenous DNA is not introduced by a retrovirus.

Another embodiment provides a method to increase stable gene targeting via exogenous DNA integration comprising inhibiting (completely or partially) expression (RNA or protein expression) of at least one gene of a DNA DSB repair pathway or by inhibiting (completely or partially) activity of at least one protein of a DNA DSB repair pathway so as to provide increased DNA integration as compared to a cell in which expression and/or activity has not been inhibited.

In one embodiment, the DNA DSB repair pathway is the C-NHEJ pathway. In another embodiment, the DNA DSB repair pathway is the A-NHEJ pathway.

In one embodiment, the gene is selected from the group consisting of Ku70, Ku86, DNA-PKcs, Artemis, LIGIV, XLF, XRCC4 or a combination thereof. In another embodiment, the gene is selected from the group consisting of Artemis, LIGIV, XLF, XRCC4 or a combination thereof. In one embodiment, the gene is not Ku70, K986 or DNA-PKcs.

In one embodiment, the gene is selected from the group consisting of LIGIII, PARP1, RAD54B, XRCC1, XRCC3, MRE11, NBS1, RAD50, CtIP, FEN1, EXO1, BLM or a combination thereof.

In one embodiment, the expression is transiently inhibited and in another embodiment, the expression permanently inhibited.

In one embodiment, the protein is selected from the group consisting of Ku70, Ku86, DNA-PKcs, Artemis, LIGIV, XLF, XRCC4 or a combination thereof. In another embodiment, the protein is selected from the group consisting of Artemis, LIGIV, XLF, XRCC4, or a combination thereof. In one embodiment, the protein is not Ku70, K986 or DNA-PKcs.

In one embodiment, the protein is selected from the group consisting of LIGIII, PARP1, RAD54B, XRCC1, XRCC3, MRE11, NBS1, RAD50, CtIP, FEN1, EXO1, BLM, or a combination thereof.

In one embodiment, the protein is inhibited by a small molecule such as NU47712, wortmannin, NU7026, vanillin or a combination thereof. In one embodiment, DNA-PKcs is inhibited by a small molecule inhibitor selected from the group consisting of NU7441, wortmannin, NU7026, vanillin or a combination thereof. In another embodiment, DNA-PKcs is inhibited by a small molecule inhibitor selected from the group consisting of wortmannin, NU7026, vanillin or a combination there.

In one embodiment, the telomeres are not dysfunctional. In another embodiment, the gene integration and or targeting is mediated by a retrovirus, rAAV, dsDNA, ssDNA, zinc finger nuclease, homing nuclease, meganuclease, transcription activator like (TAL) effector nuclease or a combination thereof.

One embodiment provides a method for increasing gene targeting frequency (as compared to current protocols or as compared to a method without inhibition) comprising inhibiting (completely or partially) expression of Artemis (RNA or protein expression) or activity of Artemis protein. Another embodiment provides a method for increasing targeted DNA integration (compared to current protocols or as compared to a method without inhibition) comprising inhibiting (completely or partially) expression of Artemis (RNA or protein expression) or activity of Artemis protein.

Another embodiment provides a method to reduce stable random DNA (e.g., exogenous) integration comprising inhibiting (completely or partially) expression (RNA or protein expression) of Artemis or by inhibiting (completely or partially) activity of Artemis protein so as to provide decreased exogenous DNA integration as compared to a cell in which expression and/or activity has not been inhibited.

Another embodiment provides a method to increase stable gene targeting via exogenous DNA integration comprising inhibiting (completely or partially) expression (RNA or protein expression) of Artemis or by inhibiting (completely or partially) activity of Artemis protein so as to provide increased DNA integration as compared to a cell in which expression and/or activity has not been inhibited.

In one embodiment, the inhibiting and integration is carried out by contacting a cell with an agent (e.g. small molecule or nucleic acid (e.g., siRNA)) so as to inhibit gene expression and/or protein activity and/or contacting a cell with the nucleic acid to be integrated (via viral, for example, rAAV, or non-viral methods, as are known and available to an art worker).

### BRIEF DESCRIPTION OF THE DRAWINGS

FIG. 1: The permanent reduction of DNA-PKcs expression results in increased rAAV-mediated gene targeting frequencies. Either wild-type (WT), DNA-PK_{cs}^{+/-}(+/−) or DNA-PK_{cs}^{-/-}(−/−) HCT116 cell lines were infected with rAAV vectors that targeted either the Ku70 or the C—C chemokine receptor type 5 (CCR5) loci. The cells were then aliquoted by limiting dilution into 96-well plates and placed under G418 selection (1 mg/mL). The total numbers of correctly targeted colonies were determined by both 5′- and 3′ diagnostic PCRs and the total number of stably integrated viruses determined by scoring the number of G418-resistant colonies and confirmed by PCR. The data for Ku70 targeting were: WT, 3/437 (0.7%); +/−, 9/163 (5.5%); −/−, 5/74 (6.8%). The data for CCR5 targeting were: WT 3/262 (1.2%); +/−4/62 (6.5%); −/−14/152 (9.2%).

FIG. 2: The transient reduction of DNA-PK_{cs} protein expression by RNAi results in increased rAAV-mediated gene targeting frequencies. Wild-type HCT116 cells were treated via transfection with RNAi-directed against DNA-PK_{cs}. At 48 hr post-transfection the level of DNA-PK_{cs} as determined by Western immunoblotting analysis was only 1% of that of the cells at the start of the experiment (FIG. 2A). Cells at this time point were infected with a rAAV vector that targeted the CCR5 locus. The total number of correctly targeted clones was 11 from 109 total G418-resistant clones for a correct gene targeting frequency of 10.1% (FIG. 2B).

FIG. 3: The transient reduction of DNA-PK_{cs} activity by NU7441 results in increased rAAV-mediated gene targeting frequencies. At time 0 HCT116 cells were infected with a rAAV vector that targeted the CCR5 locus. Also at time 0, the cells were treated with 10 μM of NU7441, an inhibitor of the DNA-PK_{cs} kinase activity, and then again at 4 hr post-infection. At the indicated times, whole cell extracts were prepared from a portion of the cells and assayed for DNA-PK complex kinase activity using a standard peptide assay. As a control, a peptide derived from p53 that is a good (+) substrate for DNA-PK was used as well as a mutated p53 peptide that is a poor (−) substrate. As additional controls, whole cell extracts were prepared from MO59J and MO59K cells, which are known to be deficient and proficient, respectively, for DNA-PK. These assays demonstrated that NU7441 nearly completely ablated DNA-PK activity, but that this inhibition was quite transient, lasting only for a matter of hours (FIG. 3A). Approximately two weeks following infection, individually G418-drug resistant clones were analyzed for correct targeting using diagnostic PCR assays. The total number of correctly targeted clones was 17 from 229 total G418-resistant clones for a correct gene targeting frequency of 7.4% (FIG. 3B).

FIG. 4: The absence of C-NHEJ factors results in decreased random, stable integration frequencies. Wild-type (WT), LIGIV-null (LIGIV), and DNA-PKcs-null (DNA-PKcs) HCT116 cells were transfected with either linearized pcDNA3.1 (which confers resistance to G418) or pPuromycin (pPur) plasmids and two weeks later, the relative number of drug-resistant colonies was determined.

FIG. 5: The absence of DNA-PKcs results in increased retroviral stable integration frequencies. Wild-type (WT), LIGIV-null (LIGIV), DNA-PKcs-null (DNA-PKcs) or XRCC3-null (XRCC3) HCT116 cells were infected with either pLPC (which confers resistance to puromycin) or HIV:GFP (HIV) and either two weeks or 3 days, respectively, later the number of puromycin-resistant colonies of GFP-positive cells, respectively, was determined.

### DETAILED DESCRIPTION OF THE INVENTION

Using genetics (mutant cell lines), molecular biology (e.g., RNAi/shRNA) and biochemistry (chemical inhibitors), genes are identified that modulate gene targeting, such as viral (rAAV), ssDNA, dsDNA, meganuclease, TAL and Zn-finger mediated gene targeting. Since gene targeting is a direct result of the balance between homologous recombination and NHEJ-mediated random integration, the present invention is generally directed, in part, towards methods, mechanisms, compositions, and kits for initiating, modulating, and/or stimulating homologous recombination. Simultaneously, the present invention improves targeted integrations by decreasing the randomness of undesired, non-targeted integrations. The methods of the invention provide elevated frequencies of correct gene targeting, including from about 5 to 10-fold increase or greater in correct gene targeting, from, for example, viral mediated gene targeting. Also, provided herein is the identification of genes that can decrease random DNA integration (the incoming DNA becoming one with the chromosomal DNA by covalent integration).

The invention may be used for any purpose including, for example, research, therapeutics, and generation of cell lines or transgenic animals (e.g., non-human animals such as mice, rats, guinea pigs, domestic animals etc.). The cells and transgenic animals may be used in gene therapy or to study gene structure and function or biochemical processes. In addition, the transgenic mammals may be used as a source of cells, organs, or tissues, or to provide model systems for human disease.

### DEFINITIONS

As used herein, the terms below are defined by the following meanings:

“Host organism” is the term used for the organism in which gene targeting, according to the invention, is carried out. “Host cell” or “target cell” refers to a cell to be transduced/transfected with a specific viral vector/nucleic acid. The cell is optionally selected from in vitro cells such as those derived from cell culture, ex vivo cells, such as those derived from an organism, and in vivo cells, such as those in an organism. “Cells” include cells from, or the “subject” is, a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird. “Cell line” refers to individual cells, harvested cells and cultures containing cells. A cell line can be continuous, immortal or stable if the line remains viable over a prolonged period of time, such as about 6 months. “Cell line” can also include primary cell cultures. Cells which may be subjected to gene targeting may be any mammalian cells of interest, and include both primary cells and transformed cell lines, which may find use in cell therapy, research, interaction with other cells in vitro or the like.

“Target” refers to the gene or DNA segment or nucleic acid molecule, subject to modification by the gene targeting method of the present invention. Generally, the target is an endogenous gene, coding segment, control region, intron, exon, or portion thereof, of the host organism. The target can be any part or parts of genomic DNA.

“Target gene modifying sequence” is a DNA segment having sequence homology to the target, but differing from the target in certain ways, in particular, with respect to the specific desired modification(s) to be introduced in the target.

“Marker” is the term used herein to denote a gene or sequence whose presence or absence conveys a detectable phenotype of the organism. Various types of markers include, but are not limited to, selection markers, screening markers, and molecular markers. Selection markers are usually genes that can be expressed to convey a phenotype that makes the organism resistant or susceptible to a specific set of conditions. Screening markers convey a phenotype that is a readily observable and a distinguishable trait. Molecular markers are sequence features that can be uniquely identified by oligonucleotide or antibody probing, for example, RFLP (restriction fragment length polymorphism), SSR markers (simple sequence repeat), epitope tags and the like.

The term “isolated” refers to protein(s)/polypeptide(s), nucleic acid(s)/oligonucleotide(s), factor(s), cell or cells which are not associated with one or more protein(s)/polypeptide(s), nucleic acid(s)/oligonucleotide(s), factors, cells or one or more cellular components that are associated with the protein(s)/polypeptide(s), nucleic acid(s)/oligonucleotide(s), factor(s), cell or cells in vivo.

An “effective amount” generally means an amount that provides the desired local or systemic effect and/or performance.

As used herein, “fragments,” “analogues” or “derivatives” of the polypeptides/nucleotides described include those polypeptides/nucleotides in which one or more of the amino acid residues are substituted with a conserved or non-conserved amino acid residue and which may be natural or unnatural. In one embodiment, variant, derivatives and analogues of polypeptides/nucleotides will have about 70% identity with those sequences described herein. That is, 70% of the residues are the same. In a further embodiment, polypeptides/nucleotides will have greater than 75% identity. In a further embodiment, polypeptides/nucleotides will have greater than 80% identity. In a further embodiment, polypeptides/nucleotides will have greater than 85% identity. In a further embodiment, polypeptides/nucleotides will have greater than 90% identity. In a further embodiment, polypeptides/nucleotides will have greater than 95% identity. In a further embodiment, polypeptides/nucleotides will have greater than 99% identity.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the disclosures of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12:387 (1984)), BLASTP, BLASTN and FASTA (Altschul, S. F. et al., J. Molec. Biol., 215:403 (1990)). The BLASTX program is publicly available from NCBI and other sources {BLAST Manual, Altschul, S. et al., NCVI NLM NIH Bethesda, Md. 20894, Altschul, S. F. et al., J. Molec. Biol., 215:403 (1990), the disclosures of which are incorporated herein by reference}. These programs optimally align sequences using default gap weights in order to produce the highest level of sequence identity between the given and reference sequences. As an illustration, by a polynucleotide having a nucleotide sequence having at least, for example, 95% “sequence identity” to a reference nucleotide sequence, it is intended that the nucleotide sequence of the given polynucleotide is identical to the reference sequence except that the given polynucleotide sequence may include up to 5 point mutations per each 100 nucleotides of the reference nucleotide sequence. In other words, in a polynucleotide having a nucleotide sequence having at least 95% identity relative to the reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95% sequence identity to a reference amino acid sequence, it is intended that the given amino acid sequence of the polypeptide is identical to the reference sequence except that the given polypeptide sequence may include up to 5 amino acid alterations per each 100 amino acids of the reference amino acid sequence. In other words, to obtain a given polypeptide sequence having at least 95% sequence identity with a reference amino acid sequence, up to 5% of the amino acid residues in the reference sequence may be deleted or substituted with another amino acid, or a number of amino acids up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions that are not identical differ by conservative amino acid substitutions.

General methods regarding polynucleotides and polypeptides are described in: Sambrook et al., Molecular Cloning: A Laboratory Manual, 2nd ed., Cold Spring Harbor, N.Y., 1989; Current Protocols in Molecular Biology, edited by Ausubel F. M. et al., John Wiley and Sons, Inc. New York; PCR Cloning Protocols, from Molecular Cloning to Genetic Engineering, Edited by White B. A., Humana Press, Totowa, N.J., 1997, 490 pages; Protein Purification, Principles and Practices, Scopes R. K., Springer-Verlag, New York, 3rd Edition, 1993, 380 pages; Current Protocols in Immunology, edited by Coligan J. E. et al., John Wiley & Sons Inc., New York, which are herein incorporated by reference.

Methods involving gene targeting with parvovirus' including adeno-associate virus (AAV) are described in, for example, WO 98/48005 and WO 00/24917, which are incorporated herein by reference. Other methods involving gene targeting are disclosed in, for example, U.S. Pat. Nos. 6,528,313 and 6,528,314, which are incorporated herein by reference. Additional methods are described in Kohli et al., Nucl. Acids Res., 32:e3 (2004) and then modified by Topaloglu et al., Nucl Acids Res., 33:e158 (2005), Konishi et al., Nat. Protoc., 2:2865 (2007), Rago et al., Nat. Protoc., 2:2734 (2007), Zhang et al., Nat. Meth., 5:163 (2008) or Berdougo et al., Meth. Mol. Biol., 545:21 (2009), which are incorporated herein by reference.

The terms “comprises,” “comprising,” and the like can have the meaning ascribed to them in U.S. Patent Law and can mean “includes,” “including” and the like. As used herein, “including” or “includes” or the like means including, without limitation.

### DNA DSB Repair/Gene Targeting

Somatic gene targeting is defined as the intentional modification of a specific genetic locus in a living cell. This technology has three general applications of interest. One is the inactivation of genes (“knockouts”), a process in which the two wild-type alleles of a gene are sequentially, or contemporaneously, inactivated in order to determine the loss-of-function phenotype(s) of that particular gene. The second application (“knock-ins”) is the subtle alteration of either one or both wild-type alleles of a gene in order to determine a partial loss-of-function, or gain-of-function, phenotype(s) and/or to affix an epitope, or reporter gene, onto the gene of interest. Most knock-ins involve the introduction of point mutations into the gene such that one and only one amino acid is altered, which then allows the role of that amino acid—in the context of the whole protein—to be determined. The third application (“gene therapy”) is technically also a “knock-in,” but the biological intent is reversed. Thus, instead of taking a wild-type gene and trying to introduce a mutation or epitope into it, as is done in a standard knock-in approach, in gene therapy one attempts to correct a preexisting mutated allele of a gene back to wild-type in order to alleviate some pathological phenotype associated with the mutation. While these three applications have conceptually different biological outcomes, they are mechanistically similar, as all appear to proceed through a form of DNA DSB repair termed homologous recombination (HR).

C-NHEJ is an evolutionarily conserved process that joins nonhomologous DNA molecules together. In their work on gene targeting, Thomas and Capecchi (Cell, 1987, 51:503-510) showed that although somatic mammalian cells can integrate a linear duplex DNA into corresponding homologous chromosomal sequences using HR, the frequency with which recombination into nonhomologous sequences occurred via C-NHEJ was at least 1,000-fold greater. Although not all of the details of C-NHEJ have been elucidated, much is known about the process. First, the heterodimeric Ku (Ku86:Ku70) protein binds onto the ends of the donor DNA and prevents the nucleolytic degradation that would otherwise shunt the DNA into the HR pathway. The binding of Ku to the ends of the DNA then recruits and activates the DNA-dependent protein kinase complex catalytic subunit (DNA-PKcs). This DNA:protein complex is then brought into contact with a chromosome into which a DSB is introduced by a mechanism that is poorly understood, although it correlates frequently with chromosomal palindromic sequences. Regardless, the chromosomal ends are probably also occupied by Ku and DNA-PKcs, which facilitates the formation of a synaptic complex with the donor DNA. Once DNA-PKcs is properly assembled at the broken ends, it, in turn, recruits additional factors such as the nuclease, Artemis, to trim the ends and a trimeric DNA ligase complex consisting of DNA ligase IV (LIGIV):x-ray cross-complementing group 4 (XRCC4):XRCC-4-like factor (XLF), to seal the break(s). In summary, mammals, such as humans, are different from bacteria and lower eukaryotes in that DSB repair proceeds primarily through a C-NHEJ recombinational pathway. Moreover, C-NHEJ must be overcome to facilitate gene targeting, which can only occur when the incoming DNA is shunted into the HR pathway.

This description of DSB repair pathways is complicated by the existence of a subpathway of NHEJ, termed alternative-NHEJ (A-NHEJ) that proceeds in a completely Ku-independent manner. And in contrast to C-NHEJ, the mechanism of, and the factors involved in, A-NHEJ remain elusive. Mechanistically, it is believed that during A-NHEJ both ends are resected 5′-to-3′ on one strand (in a process that is perhaps regulated by Mre11:Rad50:Nbs1 (MRN)) to generate 3′-single-stranded overhangs containing regions of microhomology, which then mediate the repair event. Because of this reaction pathway, deletion of the sequences between the microhomologies occurs as does deletion of one of the blocks of (micro)homology. The remaining block of microhomology always remains at the site of repair and can be used as a landmark to define A-NHEJ events. A-NHEJ was thought to be an irrelevant DSB repair pathway because it could only be detected in the absence of C—NHEJ. Interest in A-NHEJ increased with the demonstration that A-NHEJ could substitute for C-NHEJ during class switch recombination. Moreover, microhomology has been found at the junctions of ionizing radiation-induced genomic rearrangements implying that even clinically relevant DSBs can be repaired by A-NHEJ. Lastly, microhomologies are detected at breakpoints for chromosomal deletions and translocations in human cancer cells. These observations have propelled many laboratories to identify the A-NHEJ factors. These studies have implicated DNA ligase III (LIGIII), X-ray cross complementing 1 (XRCC1), poly (ADP-ribose) polymerase 1 (PARP1) and the MRN complex. However, additional factors may be involved.

There are three widely-accepted pathways of DNA DSB repair: HR, C-NHEJ and A-NHEJ. Experiments designed to test the impact of loss-of-function manipulations of the genes in these pathways, with a particular interest in how they impact rAAV gene targeting, have been initiated. Adeno-associated virus (AAV) is a nonpathogenic parvovirus—with a natural tropism for human cells—that is dependent upon a helper virus (usually adenovirus and hence the name) for a productive infection. In the intervening decade since it was demonstrated that recombinant AAV (rAAV) could be used as a vector for gene targeting in human cells, this methodology has gained wide acceptance. Ninety different genes have been modified (generally knocked-out) in forty-seven different immortalized and normal diploid human cell lines. Lastly, over 20 clinical gene therapy trials utilizing rAAV are currently in progress. A better understanding of the mechanism of rAAV-mediated gene targeting and the factors that influence the frequency with which it correctly targets (presumably HR-mediated) versus those that influence its random integration (presumably C- and/or A-NHEJ-mediated) is needed.

Using rAAV-mediated gene targeting, it has been has demonstrated that the C-NHEJ genes Ku70 and Ku86 are essential in human somatic cells (Fattah et al. PNAS, 2008, 105:8703-8708; Wang et al., PNAS, 2009, 106:12430-12435). In the course of these studies, it was discovered that a reduction in the levels of Ku in human somatic cells resulted in higher (5- to 10-fold) frequencies of rAAV-mediated correct gene targeting (Fattah et al. PNAS, 2008, 105:8703-8708). In particular, RNA interference and short-hairpinned RNA strategies to deplete Ku70 in wild-type cells phenocopied the genetic inactivation of a Ku70 allele and greatly accentuated them in Ku70^{+/-} cell lines at three independent loci. These data demonstrated that gene-targeting frequencies can be significantly improved by impairing the C-NHEJ pathway and we proposed that Ku70-depletion could be used to facilitate knockout, knock-in and gene therapy approaches.

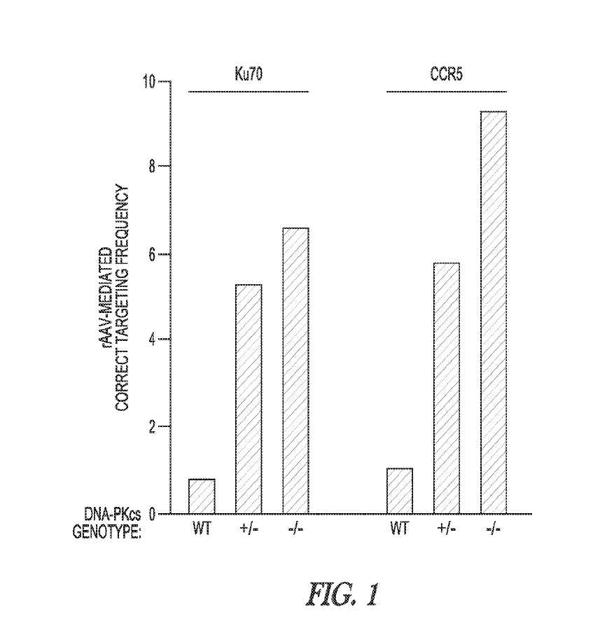

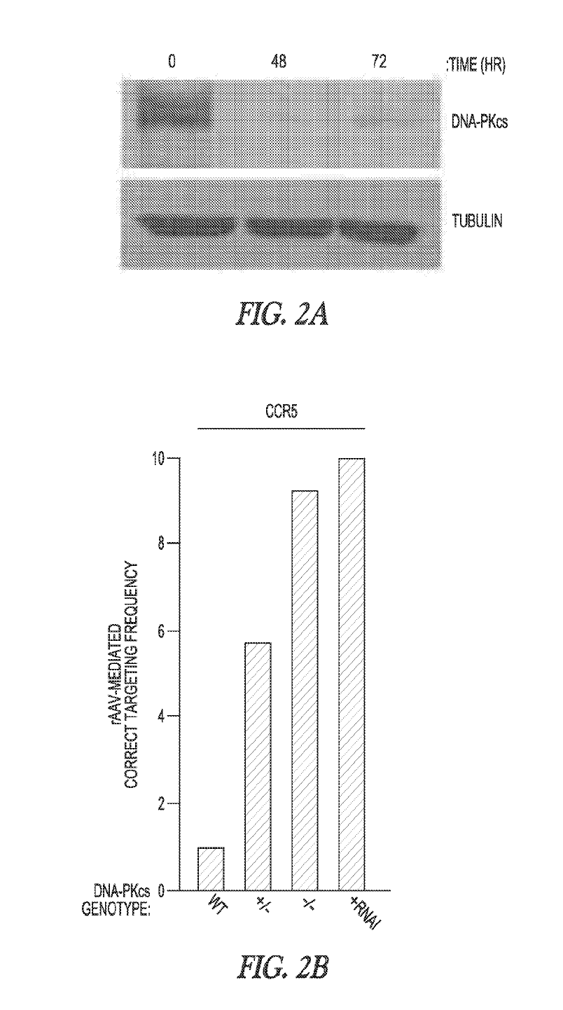

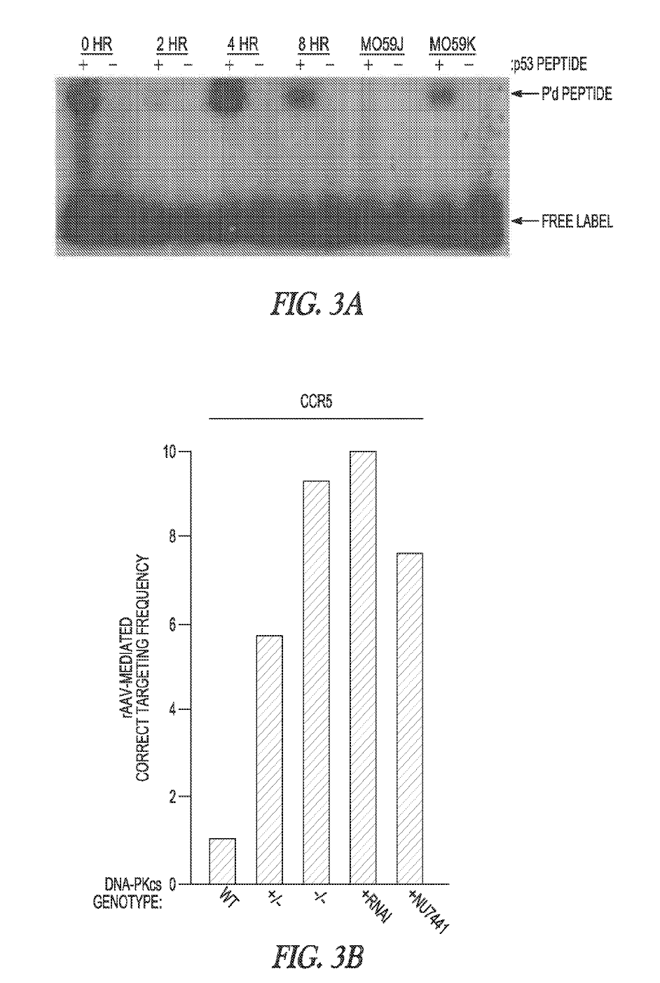

Unfortunately, it was demonstrated that the prolonged absence of Ku results in telomere dysfunction that is so severe that it is not compatible with viability (Wang et al., PNAS, 2009, 106:12430-12435). To extend these observations, a series of human HCT116 cell lines defective in genes involved in the three pathways of DNA DSB repair have been generated. One of these HCT116 cell lines contained loss-of-function mutations in either one or both alleles of DNA-PK_{cs} (Ruis et al., Mol. Cell. Biol., 2008, 28:6182-6195). With rAAV vectors constructed to target the chemokine-receptor 5 (CCR5) locus, it was demonstrated that these cells exhibited a 5- and 10-fold increase, respectively, in rAAV-mediated gene targeting frequencies compared to wild-type cells (FIG. 1). Thus, NHEJ can compete with HR in cells and, in the absence of C-NHEJ, HR can become the preferred mechanism for DNA DSB repair. Unfortunately, the loss of DNA-PK_{cs} similar to the loss of Ku, causes profound genomic instability due to telomere defects (Ruis et al., Mol. Cell. Biol., 2008, 28:6182-6195). To address this issue, C-NHEJ was transiently inactivated by means of knocking down DNA-PK_{cs} with RNAi using commercially available SMARTPool™ reagents from Dharmacon RNA Technologies. DNA-PK_{cs} expression was reduced for several days (FIG. 2A) and when the cells were infected with rAAV CCR5 gene targeting vectors during this time frame the frequency of gene targeting increased ˜10-fold (FIG. 2B). When several of the correctly targeted clones were analyzed by G-band karyotyping they showed no evidence of telomere loss and/or genomic instability. Thus, in contrast to the permanent loss of DNA-PK_{cs} expression, a transient reduction of DNA-PK_{cs} seems to be tolerated well by the cells while simultaneously facilitating enhanced gene targeting. These experiments were supported by parallel studies in which DNA-PK_{cs} kinase activity was inhibited with a small molecule inhibitor NU7441 in order to phenocopy the high targeting frequencies of DNA-PK_{cs}-null cells without occurring the telomere defect mediated by genomic instability. NU7441 transiently inhibited DNA-PK_{cs} for a period of several hours (FIG. 3A) and when the cells were infected with rAAV CCR5 gene targeting vectors during this time frame the frequency of gene targeting increased ˜7-fold (FIG. 3B). Taken together, these experiments demonstrated that it was technically feasible to enable rAAV-mediated gene targeting frequencies that were similar if not higher compared to those of DNA-PK_{cs} null cells while preserving genomic stability. Thus, the inactivation of NHEJ appears to create a “window of opportunity” that can be an effective approach to gene therapy.

Genes and proteins involved in NHEJ can include, but are not limited to, Ku70 (NM_{—}001469; NP_{—}001460); Ku86 (NM_{—}021141; NP 066964); DNA-PKcs (NM_{—}006904; NP 008835); Artemis (NM_{—}001033858; NP 001029030); LIGIV (NM_{—}002312; NP_{—}002303); XLF (NM_{—}024782; NP079058); XRCC4 (NM_{—}022550; NP 072044); LIGIII (NM_{—}013975; NP 039269); PARP1 (NM_{—}001618; NP 001609); RAD54B (NM_{—}012415; NP 036547); XRCC1 (NM_{—}006297; NP 006288); XRCC3 (NM_{—}001100118; NP 001093588); MRE11 (NM_{—}005591; NP 005582); NBS1 (NM_{—}002485; NP 002476); RAD50 (NM_{—}005732; NP 005723); CtIP (U72066; AAC14371); FEN1 (NM_{—}004111; NP 004102); EXO1 (NM_{—}130398; NP 569082); BLM (NM_{—}000057; NP 000048) or variants thereof (each accession number is incorporated herein by reference).

a. rAAV Mediated Gene Targeting

AAV is a small, nonenveloped, single-stranded DNA (ssDNA) virus belonging to the Parvoviridae family. It is estimated that 80⁺% of the population is seropositive for AAV, however there is no evidence of any association of disease or pathology with AAV. Of the many identified AAV serotypes, type 2 (AAV-2) is the one most commonly isolated from humans and it is this serotype that has been used predominately by basic scientists and most clinicians. AAV-2 is a nonpathogenic parvovirus with a natural tropism for human cells that depends on a helper virus (usually adenovirus and hence the name) for a productive infection. The AAV-2 genome is encapsidated as a ssDNA molecule of 4.6 kb flanked by 145 bp long inverted terminal repeat (ITR) sequences. The recombinant form of AAV (rAAV) is constructed by replacing the AAV-2 genome with a gene(s) or sequence(s) of interest between the two ITRs. With a packaging capacity up to 4.9 kb, rAAV vectors can then be produced into human cells by co-transfecting the rAAV plasmid along with an AAV helper plasmid containing the replication (Rep) and capsid (Cap) genes.

In the intervening decade since it was demonstrated that rAAV could be used as a vector for gene targeting in human cells, this methodology has gained wide acceptance. To date, there have been 684 rAAV-mediated correctly-targeted events recorded in the literature from a total of 22,446 viral integrations. This overall targeting frequency of 3.0% is better than traditional transfection-based approaches. Moreover, the rAAV methodology is both simple and expeditious; the entire experiment to knock out a gene can take as little as 2 months and although it usually takes 4 to 6 months this is still significantly faster than any competing methodology. Kohli et al. (Nucl. Acids Res., 2004, 32:e3) developed a protocol driven almost exclusively by PCR to construct the targeting vectors and viral stocks, thus enhancing the ease of working with rAAV. Additionally, the rAAV targeting (homology) arms are short enough (<1.0 kb) to enable screening the resulting clones by PCR instead of Southern blots, once again expediting the targeting process. The viral vectors and helper plasmids (expressing the necessary viral packaging factors) are all commercially available (Stratagene).

b. Nucleases

Zinc-finger nucleases (ZFNs) are artificial restriction enzymes generated by fusing a zinc finger DNA-binding domain to a DNA-cleavage (the FokI nuclease) domain. The zinc finger domains can be engineered to target desired DNA sequences, which then enables the nuclease domain to cleave unique sequences within a complex genome. By taking advantage of the endogenous DNA DSB repair machinery, ZFN reagents can be used to precisely alter the genomes of higher organisms.

The non-specific nuclease domain from the type II restriction endonuclease FokI is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a defined distance apart. The most commonly used linker sequences between the zinc finger domain and the cleavage domain requires the 5′ edge of each binding site to be separated by 5 to 7 bp.

The DNA-binding domains of individual ZFNs typically contain between three and six individual zinc finger repeats and thus each DNA binding domain recognizes between 9 and 18 basepairs. Various strategies have been developed to engineer zinc fingers to bind desired sequences. These include both “modular assembly” and selection strategies that employ either phage display or cellular selection systems.

The most straightforward method to generate new zinc-finger arrays is to combine smaller zinc-finger “modules” of known specificity. The most common modular assembly process involves combining three separate zinc fingers that can each recognize a 3 basepair DNA sequence to generate a 3-finger array that can recognize a 9 basepair target site. Other procedures can utilize either 1-finger or 2-finger modules to generate zinc-finger arrays with six or more individual zinc fingers.

Numerous selection methods have been used to generate zinc-finger arrays capable of targeting desired sequences. Initial selection efforts utilized phage display to select proteins that bound a given DNA target from a large pool of partially randomized zinc-finger arrays. More recent efforts have utilized yeast one-hybrid systems, bacterial one-hybrid and two-hybrid systems, and mammalian cells.

ZFNs have become useful reagents for manipulating genomes of many higher organisms including Drosophila melanogaster, Caenorhabditis elegans, sea urchin, tobacco, corn, zebrafish, and various types of mammalian cells (e.g., human). ZFNs can be used to disable dominant mutations in heterozygous individuals by producing DSBs in the DNA in the mutant allele which will, in the absence of a homologous template, be repaired by NHEJ, which is inherently error-prone. Multiple pairs of ZFNs can also be used to completely remove entire large segments of genomic sequence.

ZFNs are also used to rewrite the sequence of an allele by invoking the HR machinery to repair the DSB using a supplied DNA fragment as a template. The HR machinery searches for homology between the damaged chromosome and the extra-chromosomal fragment and copies the sequence of the fragment between the two broken ends of the chromosome, regardless of whether the fragment contains the original sequence.

Using ZFNs to modify endogenous genes has traditionally been a difficult task due mainly to the challenge of generating zinc finger domains that target the desired sequence with sufficient specificity. Improved methods of engineering zinc finger domains and the availability of ZFNs from a commercial supplier now put this technology in the hands of increasing numbers of researchers. Several groups are also developing other types of engineered nucleases including engineered homing endonucleases (Grizot et al., 2009, Nucl. Acids Res., 37:5405-5419; Gao et al., 2010 Plant J., 61:176-187) and nucleases based on engineered transcription activator like (TAL) effectors (Christian et al., 2010, Genetics, 186:757-761; Li et al., 2010, Nucl. Acids Res., 39:359-372). TAL effector nucleases (TALENs) are particularly interesting because TAL effectors appear to be very simple to engineer (Moscou et al., 2009, Science 326:1501; Boch et al., 2009, Science 326:1509-1512).

Inhibition of Gene Expression and/or Protein Activity

a. Inhibition of Gene Expression

The expression of RNA and/or protein can be inhibited by a variety of methods. For example, RNA expression can be inhibited by “knockout” procedures or “knockdown” procedures. Generally, with a “knockout,” expression of the gene in an organism or cell is eliminated by engineering the gene to be inoperative or removed. In a “knockdown,” the expression of the gene may not be completely inhibited, but only partially inhibited, such as with antisense (antisense molecules interact with complementary strands of nucleic acids, modifying expression of genes), RNAi or shRNA technology.

In RNA interference (RNAi), double-stranded RNA is synthesized with a sequence complementary to a gene of interest and introduced into a cell or organism, where it is recognized as exogenous genetic material and activates the RNAi pathway. A small hairpin RNA or short hairpin RNA (shRNA) is a sequence of RNA that makes a tight hairpin turn that can be used to silence gene expression via RNA interference. Small interfering RNA (siRNA), sometimes known as short interfering RNA or silencing RNA, is a class of double-stranded RNA molecules that play a variety of roles in biology. Most notably, siRNA is involved in the RNA interference (RNAi) pathway, where it interferes with the expression of a specific gene(s). siRNA can be used to modify expression of the genes mentioned herein.

b. Small Molecules to Inhibit Expression

Small molecules can be used to inhibit the expression of a gene or the activity of a protein. For example, DNA-PK_{cs}, is inhibitable. The autophosphorylation of DNA-PK_{cs} is thought to induce a conformational change that allows end processing enzymes to access the ends of a DSB. DNA-PK_{cs} also cooperates with ATR and ATM to phosphorylate proteins involved in the DNA damage checkpoint. Thus, inhibition of DNA-PK_{cs} abrogates proper DNA DSB repair. DNA-PK_{cs} belongs to the phosphatidylinositol 3-kinase-related kinase protein family. Most PI 3-kinases are inhibited by the drugs wortmannin and LY294002. As wortmannin and LY294002 are broad inhibitors against PI 3-kinases and a number of unrelated proteins, PI 3-kinase isoform specific inhibitors are being developed. NU4771 (8-dibenzothiophen-4-yl-2-morpholin-4-yl-chromen-4-one) is a specific DNA-PK_{cs} kinase inhibitor.

Genes/proteins in involved in NHEJ and small molecules that may be used to inhibit them include, but are not limited to, for example, Ku70, Ku86, DNA-PK_{cs} (NU7441), Artemis, XLF, XRCC4, LIGIV (broad spectrum ligase inhibitors and/or specific), PARP1, 3AB (3-aminobenzamide), XRCC1, LIGIII (broad spectrum ligase inhibitors and/or specific).

### Culture Conditions

During and after the gene targeting process, the cells can be cultured in culture medium that is established in the art and commercially available from the American Type Culture Collection (ATCC), Invitrogen and other companies. Such media include, but are not limited to, Dulbecco's modified Eagle's medium (DMEM), DMEM F12 medium, Eagle's minimum essential medium, F-12K medium, Iscove's modified Dulbecco's medium, knockout D-MEM, RPMI-1640 medium, or McCoy's 5A medium. It is within the skill of one in the art to modify or modulate concentrations of media and/or media supplements as needed for the cells used. It will also be apparent that many media are available as low-glucose formulations, with or without sodium pyruvate.

Also contemplated is supplementation of cell culture medium with mammalian sera. Sera often contain cellular factors and components that are needed for cell viability. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, rat serum (RS), serum replacements, and bovine embryonic fluid. It is understood that sera can be heat-inactivated at 55-65° C. if deemed needed to inactivate components of the complement cascade. Modulation of serum concentrations, or withdrawal of serum from the culture medium can also be used to promote survival of one or more desired cell types. In one embodiment, the cells are cultured in the presence of FBS/ or serum specific for the species cell type. For example, cells can be isolated and/or expanded with total serum (e.g., FBS) concentrations of about 0.5% to about 5% or greater including about 5% to about 15%. Concentrations of serum can be determined empirically.

Additional supplements can also be used to supply the cells with trace elements for optimal growth and expansion. Such supplements include insulin, transferrin, sodium selenium, and combinations thereof. These components can be included in a salt solution such as, but not limited to, Hanks' Balanced Salt Solution™ (HBSS), Earle's Salt Solution™, antioxidant supplements, MCDB-201™ supplements, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids. Many cell culture media already contain amino acids; however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

Antibiotics are also typically used in cell culture to mitigate bacterial, mycoplasmal, and fungal contamination. Typically, antibiotics or anti-mycotic compounds used are mixtures of penicillin/streptomycin, but can also include, but are not limited to, amphotericin (Fungizone™), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be advantageously used in cell culture and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine. β-mercaptoethanol can also be supplemented in cell culture media.

Lipids and lipid carriers can also be used to supplement cell culture media, depending on the type of cell and the fate of the differentiated cell. Such lipids and carriers can include, but are not limited to cyclodextrin (α,β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulation.

Cells in culture can be maintained either in suspension or attached to a solid support, such as extracellular matrix components and synthetic or biopolymers. Cells often require additional factors that encourage their attachment to a solid support (e.g., attachment factors) such as type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin.

### EXAMPLES

The following examples are provided in order to demonstrate and further illustrate certain embodiments and aspects of the present invention and are not to be construed as limiting the scope thereof.

### Example I

### Materials and Methods

### Cell Culture

The human colon cancer cell line HCT116 was obtained from the American Type Culture Collection and maintained in McCoy's 5A media (Mediatech Inc.) containing 10% heat inactivated fetal calf serum (Cambrex), 2 mM L-glutamine, 100 U/ml penicillin and 100 U/ml streptomycin (Invitrogen). The cells were grown at 37° C. in a humidified incubator with 5% CO₂. Cell lines derived from correct targeting events were grown either in the presence of 1 mg/ml G418 or 1 μg/mL puromycin.

### Silencing of DNA-PKcs

For the RNAi experiments, pre-designed, double-stranded siRNAs targeting human DNA-PK_{cs} were purchased from Dharmacon (SMARTPool reagents; Dharmacon RNA Technologies). Specifically, 4 siRNAs were used either separately or in combination. They were: 1) GGAAGAAGCUCAUUUGAUU (SEQ ID NO:1) (J-005030-06, PRKDC), 2) GAGCAUCACUUGCCUUUAA (SEQ ID NO:2) (J-005030-07, PRKDC), 3) GCAGGACCGUGCAAGGUUA (SEQ ID NO:3) (J-005030-08, PRKDC) and 4) AGAUAGAGCUGCUAAAUGU (SEQ ID NO:4) (J-005030-09, PRKDC). As a control, a nontargeting siRNA was also used (ON_TARGETplus Non-targeting siRNA #1, D-001819-01-05). Prior to transfection, cells were seeded in a 6-well plate and then incubated in normal medium without antibiotics overnight such that they reached 30 to 40% confluence. Transfections were then performed twice with Dharmafect1 according to the manufacturer's instructions.

### Targeting Vector Construction, Packaging, and Infection

The targeting vectors, Ku70-Neo (Fattah et al., DNA Repair, 2008, 7:762-774) and CCR5-Neo (Fattah et al., PNAS, 2008, 105:8703-8708), were constructed by using the rAAV system as described (Kohli et. al., Nucl. Acids Res., 2004, 32:e3). All virus packaging and infections were performed as described (Kohli et. al., Nucl. Acids Res., 2004, 32:e3).

### Isolation of Genomic DNA and Genomic PCR

Genomic DNA for PCR screening was isolated by using phenol extraction followed by ethanol precipitation. DNA-PK_{cs}- and CCR5-targeting events were identified by PCR using conditions described elsewhere (Ruis et. al., Mol. Cell. Biol., 2008, 28:6182-6195; Kohli et. al., Nucl. Acids Res., 2004, 32:e3). The primers used to screen for DNA-PKcs targeting events were LArmR, GCTCCAGCTTTTGTTCCCTTTAG (SEQ ID NO:5) and PKcs81-83F1, CTCATACTTACTATGGATTGTGTGTATATCTACC. (SEQ ID NO:6) The primers used to screen for CCR5 targeting events were CF, GCACCATGCTTGACCCAG (SEQ ID NO:7) and NR, GTTGTGCCCAGTCATAGCCG (SEQ ID NO:8).

### Whole Cell Extract Preparation

Cells were trypsinized and washed twice with phosphate buffered saline. For whole cell extraction, cells were boiled in 10 mM Tris-HCl, pH 7.5 and 5 mM MgCl₂ containing a 2× protease inhibitor cocktail (Roche) for 10 min. The samples were then digested with DNaseI (0.1 U/μ1; Gibco) for 10 min at 37° C. The samples were finally boiled in 5×SDS buffer (0.225 M Tris-HCl, pH 6.8, 50% (v/v) glycerol, 5% SDS, 0.05% bromophenol blue, 0.14M β-mercaptoethanol) and used for immunoblotting.

### Antibodies and Immunoblotting

DNA-PKcs antibodies purchased from Calbiochem were used at a 1:50 dilution for Western blot analysis. An α-tubulin antibody (Covance Research Products) was diluted at a 1:10,000 dilution and used for a loading control. For immunoblot detection, proteins were subjected to electrophoresis on a 4-20% gradient gel (Bio-Rad), electroblotted onto a nitrocellulose membrane and detected as described (Ruis et. al., Mol. Cell. Biol., 2008, 28:6182-6195).

### DNA-PK Assays

Nuclear extracts were incubated on ice for 15 min with preswollen dsDNA-cellulose (Sigma). Nuclear extract (100 μg) was used with each sample. Following incubation on ice, the samples were washed twice in Z′ 0.05 buffer (25 mM HEPES-KOH, 50 mM KCl, 10 mM MgCl₂, 20% glycerol, 0.1% IGEPAL™, 1 mM dithiothreitol). After the washing steps, the samples were centrifuged and the precipitate was resuspended in 100 μl Z′ 0.05 buffer. The sample was then incubated at 30° C. for 15 min with either a good DNA-PK substrate peptide EPPLSQEAFADLLKK (SEQ ID NO:9) or a mutant peptide EPPLSEQAFADLLKK (SEQ ID NO:10) and [γ-³²P]ATP. The wild-type peptide can be phosphorylated by DNA-PK at the serine residue, while the mutant peptide is not recognized by DNA-PK. Following incubation, polyacrylamide gel electrophoresis was carried out. The gel was vacuum dried and exposed to X-ray film. The amount of phosphorylated peptide was quantified using a phosphorimager.

### Increased Gene Targeting Frequencies in DNA-PKcs-reduced Human Somatic Cell Lines.

Previously, using rAAV-mediated gene targeting, human HCT116 cell lines were constructed that are wild-type, heterozygous or null for DNA-PK_{cs} expression (Ruis et. al., Mol. Cell. Biol., 2008, 28:6182-6195). These three cell lines were subsequently infected either with a rAAV knockout vector for Ku70 or CCR5. Each vector carried the NEO (neomycin resistance gene) and thus productively infected cells became G418-resistant. G418-resistant colonies (generally 100 to 200) were then individually characterized for either random integration or correct integration using four diagnostic PCR reactions (Fattah et al., PNAS, 2008, 105:8703-8708). In wild-type cells the frequency of correct gene targeting for Ku70 was 0.7% and CCR51.2% (FIG. 1). In DNA-PK_{cs}^{+/-} cells, the frequency of correct gene targeting increased to 5.5% and 6.5%, respectively (FIG. 1). In DNA-PK_{cs}^{-/-} cells, the frequency of correct gene targeting increased even more, to 6.8% and 9.2%, respectively (FIG. 1). These results demonstrate that the absence of DNA-PK_{cs} significantly increases the gene-targeting frequency in multiple loci in human somatic cells.

Thus, transient inactivation of NHEJ in HCT116 cells results in about 10-fold higher frequency of gene targeting while maintaining genomic stability.

### Example II

Cell lines defective in components of the three repair pathways described above have been or will be constructed and tested for their impact on gene targeting. Specifically, mutant loss-of-function cell lines for Ku70 (Fattah et al., 2008, DNA Repair, 7:762-774; Fattah et al., PNAS, 2008, 105:8703-8708), Ku86 (Fattah et al., 2008, DNA Repair, 7:762-774; Wang et al., PNAS, 2009, 106:12430-12435), DNA-PK_{cs} (Ruis et. al., Mol. Cell. Biol., 2008, 28:6182-6195), XLF (Fattah et al., PLoS Genet., 2010, 6:e1000855), LIGIV (Fattah et al., PLoS Genet., 2010, 6:e1000855), XRCC4 (unpublished), Artemis (unpublished), and LIGIII (unpublished) have been constructed. The following cell lines are currently in construction: PARP1, RAD54B, XRCC1 and XRCC3. The cell lines to be constructed include, but are not limited to: MRE11, NBS1, RAD50, CtIP, FEN1, EXO1, and BLM. Moreover, any combination of compound mutant cell lines can be constructed in which, in a single cell line, two or more DNA DSB repair genes have been inactivated. Thus, for example, Ku70^{+/-}:LIGIV^{-/-} and Ku70^{+/-}:DNA-PK_{cs}^{-/-} cell lines have been described (Fattah et al., PLoS Genet., 2010, 6:e1000855). Additional compound cell lines already constructed include Ku86^{-/-}:RAD54B^{-/-/-} (unpublished) and Ku86^{flox/-}:LIGIV^{-/-} (unpublished) and LIGIV^{-/-}:RAD54B^{-/-/-} and Ku86^{flox/-}:LIGIII^{-/-} are currently under construction.

The impact of the loss-of-function of these genes for rAAV-mediated gene targeting has already been tested for Ku70 (Fattah et al., PNAS, 2008, 105:8703-8708, Chen I., Nature Struc. Mol. Biol., 2008, 15:699), DNA-PK_{cs} (data provided herein; FIGS. 1, 2 and 3), and LIGIV (unpublished).

In all cases, the cell lines will either be completely defective or have reduced expression of genes that play a role in one or more of the pathways involved in DSB repair. In summary, it was determined that reductions in Ku70 increase rAAV-meditated gene targeting. However, this came with an attendant drawback, which is that reduced Ku expression also correlates with telomere dysfunction and genomic instability. The genetic, molecular and biochemical data presented in this application suggests that DNA-PK_{cs} may be a more viable target for modulating rAAV-mediated gene targeting, permitting higher frequencies of gene targeting, without the attendant genomic instability. The other DNA DSB repair genes currently under investigation, or those to be investigated, may provide better enhancements to gene targeting with equivalent or even fewer deleterious side effects.

### Example III

Stable cell lines corresponding to, but not limited to, all of the loss-of-function mutants described above (Ku70, Ku86, DNA-PK_{cs}, XLF, LIGIV, XRCC4, LIGIII, Artemis, PARP1, RAD54B, XRCC1, XRCC3, MRE11, NBS1, RAD50, CtIP, FEN1, EXO1, and BLM, as well as certain compound mutants, will be established with a single copy of a transgene (plasmid A658; Porteus and Baltimore, Science, 2003, 300:763) that contains a zinc finger targeting site, which has been engineered into a defective green fluorescent protein (GFP) coding sequence. These cell lines will be subsequently co-transfected with a plasmid expressing the ZFN (plasmid M508; Urnov et al., Nature, 2005, 435:646-651) and a plasmid (plasmid A880; Urnov et al., Nature, 2005, 435:646-651) expressing a rescuing piece of GFP coding sequence. If ZFN-mediated gene targeting occurs, the defective chromosomal GFP gene will be reanimated by the rescuing GFP coding sequences. Such correct gene targeting events can be identified and quantitated by using fluorescence activated cell sorting (FACS).

### Example IV

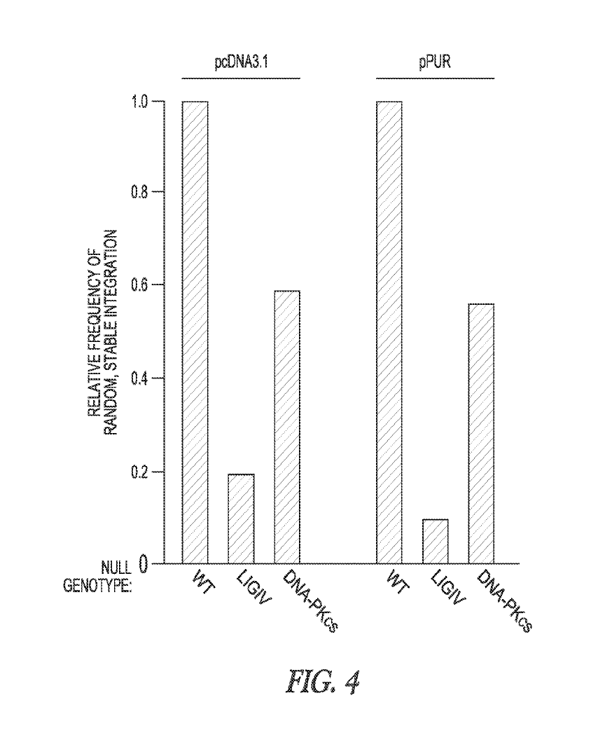

Stable cell lines corresponding to, but not limited to, all of the loss-of-function mutants described above (Ku70, Ku86, DNA-PK_{cs}, XLF, LIGIV, XRCC4, LIGIII, Artemis, PARP1, RAD54B, XRCC1, XRCC3, MRE11, NBS1, RAD50, CtIP, FEN1, EXO1, and BLM, as well as certain compound mutants, will be established and tested for their ability to stably integrate different types of DNA (e.g., linear dsDNA and retroviral DNA). For example, we have already determined that the absence of certain C-NHEJ factors decreases the frequency of stable transformation. Double-stranded DNA plasmids pcDNA3.1 (which confers resistance to G418) and pPUR (which confers resistance to puromycin) were linearized and transfected into either wild-type HCT116, LIGIV-null or DNA-PK_{cs}-null cells. Approximately two weeks later the relative frequency of drug-resistant colonies was determined. The absence of LIGIV reduced the frequency of stable transformation by 80 to 90% while the absence of DNA-PK_{cs} reduced the frequency of stable transformation by 40 to 50% (FIG. 4).

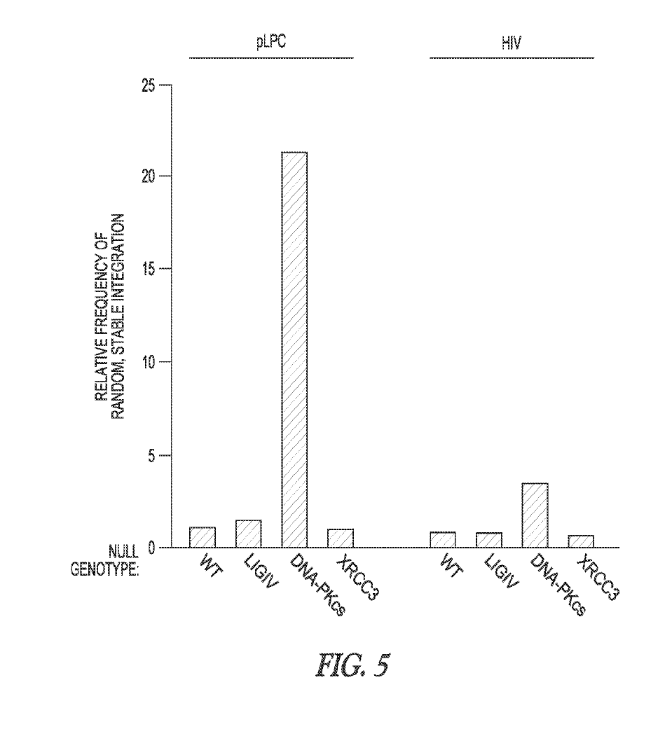

Similarly, wild-type, LIGIV-null, DNA-PK_{cs}-null or XRCC3-null cells were infected either with the pLPC retrovirus (which confers resistance to puromycin) or a HIV:GFP retrovirus (which results in GFP expression in productively infected cells) and either two weeks or three days, respectively, the percentage of puromycin-resistant or GFP-positive, respectively, cells were scored. The absence of LIGIV or XRCC3 had no statistically significant effect on retroviral integration (FIG. 5). In contrast, cells that were deficient in DNA-PKcs showed increases in retroviral transduction, although this effect was larger for pLPC than for HIV:GFP (FIG. 5). Briefly, the data suggests that the absence of specifically DNA-PK_{cs} increases retroviral transduction.

### Example IV

### rAAV Targeted Knockout of Artemis in HCT116 Cells

### Introduction

Artemis (occasionally referred to as SNMC1 (Sensitive to Nitrogen Mustard Cl)) was originally identified as a gene that, when mutated (Moshous et al.), was responsible for a subset of human patients afflicted with RS-SCID (Radiation-Sensitive, Severe Combined Immune Deficiency) (Nicolas et al.). Subsequent biochemical characterization of Artemis demonstrated that it was a DNA-PKcs-(DNA-dependent Protein Kinase complex Catalytic Subunit) dependent, structure specific nuclease (Kurosawa and Adachi). Artemis' role in causing SCID when it is mutated is well understood. Artemis has hairpin resolving nuclease activity and hairpin resolution is an intermediate step in V(D)J {Variable(Diversity)Joining} recombination, a lymphoid-restricted, site-specific recombination process in the development of the human immune system (Ma et al.). Thus, when Artemis is mutated, hairpinned V(D)J recombination intermediates accumulate and no functional B- or T-cells can be generated (Rooney et al.). Artemis' role in causing RS when it is mutated is less well understood, but presumably is due to the lack of resolution of hairpinned-like DNA structures that may be generated during ionizing radiation exposure. Interestingly, although Artemis is a member of a family of structure-specific nucleases consisting of at least five members (Cattell et al. and Yan et al.), these proteins have apparently evolved distinct properties since the expression of the other four nucleases is not sufficient to compensate for the loss of Artemis (Moshous et al.).

Although Artemis has been investigated predominately for its roles in V(D)J recombination and DNA repair, it has also been implicated in rAAV infections, but not in rAAV-mediated gene targeting. Studies carried out in either DNA-PKcs- or Artemis-deficient mouse cells showed that rAAV replication intermediates containing unprocessed hairpinned ITRs (Inverted Terminal Repeats) accumulated (Inagaki et al.) in a manner highly reminiscent of what had been observed for hairpinned V(D)J recombination intermediates (Rooney et al.). In a somewhat parallel study, the DNA locations where rAAV randomly integrates in mouse cells were identified and sequenced. These sites were biased toward palindromic (i.e., potentially hairpinned) sequences (Inagaki et al.). Thus, a model based upon these results is that Artemis may be required to process either the viral ITRs or genomic hairpins (or both) to facilitate random rAAV integrations. The bias towards genomic palindromic sequences was not observed when a similar experiment using AAV was carried out in human somatic cells (Miller et al.).

To experimentally test the hypothesis that Artemis may regulate the frequency of rAAV-mediated gene targeting, using rAAV-mediated gene targeting technology, a human somatic cell line that no longer expresses Artemis was generated. The frequency of subsequent rAAV-mediated gene targeting in this cell line was enhanced. This observation suggests that Artemis normally suppresses rAAV-mediated gene targeting. This study combined with the inventor's previous observations demonstrating an increased frequency of gene targeting in Ku and DNA-PKcs mutant cells, suggests that inhibition of C-NHEJ factors may be a generally applicable methodology to improve gene targeting, such as rAAV-mediated gene targeting.

### Materials and Methods

### Targeting Vector Construction

Construction of the pAAV-Artemis exon 2 Neo or pAAV-Artemis exon 2 Puro targeting vectors was carried out by PCR followed by restriction enzyme digestion and subsequent DNA ligation (Kohli et al.). Briefly, HCT 116 genomic DNA was used as a template for PCR reactions to create homology arms flanking exon 2 of the Artemis locus. Primers used to create either the left or right homology arms include ART2F: 5′-ATACATACGCGGCCGCGAGCCACCATGTCCAACTGGTTTAG-3′ (SEQ ID NO:10); ART2 SacIIR: TTATCCGCGGTGGAGCTCCAGCTTTTGTTCCCTTTAGAAAAGAACAAAAACTCATG AATATG-3′ (SEQ ID NO:11); ART2 KpnIF: 5′-ATGGTACCCAATTCGCCCTATAGTGAGTCGTATTACTATTTTGCTACTTGTGTTTTTA AG-3′ (SEQ ID NO:12); and ART 2R: 5′-ATACATACGCGGCCGCGTCAATAAGTAAATACAAATAAAGTAATAAAAAATTATTG GC-3′ (SEQ ID NO:13). Fusion PCR was then performed using the PCR-generated left and right homology arms along with a PvuI restriction enzyme fragment derived from the pNeDaKO vector to create a NotI digestible vector fragment that was subsequently ligated into pAAV-MCS. In addition to pAAV-Artemis exon 2 Neo, pAAV-Artemis exon 2 Puro was also created. This was achieved using the original pAAV-Artemis exon 2 Neo vector and swapping out the drug selection cassettes. Briefly, a puromycin selection cassette from an engineered pNeDaKO Puro plasmid was removed using restriction enzyme digestion with SpeI and KpnI. This DNA fragment was then ligated to the SpeI/KpnI pAAV-Artemis exon 2 homology arm-containing fragment to generate pAAV-Artemis exon 2 Puro.

### Virus Production

rAAV-Artemis Exon 2 Neo virus was generated using a triple transfection strategy in which the targeting vector (8 μg) was mixed with pAAV-RC and pAAV-helper (8 ps each) and was then transfected into 4×10⁶ AAV-293 cells using Lipofectamine 2000 (Invitrogen). Virus was isolated from the AAV-293 cells 48 hr later by scraping the cells into 1 ml media followed by three rounds of freeze/thawing in liquid nitrogen (Khan et al. and Kohli et al.).

### Infections

HCT116 cells were grown to ˜70-80% confluency on 6-well tissue culture plates. Fresh media (1 ml) was added at least 30 min prior to the addition of virus. At that time, the required amount of virus was added drop-wise to the plates. The cells and virus were allowed to incubate for 2 hr before adding back more media (3 ml). The infected cells were allowed to grow for 2 days before they were trypsinized and plated at 2000 cells per well of 96-well plates under the appropriate drug selection (Ruis et al.).

### Isolation of Genomic DNA and PCR

Genomic DNA for PCR was isolated using the PureGene DNA purification kit (Qiagen). Cells were harvested from confluent wells of a 24-well tissue culture plate. DNA was resuspended in 50 μl hydration solution, 2 μl of which was used for each PCR reaction. For Artemis exon 2 heterozygous targeting events, a control PCR was performed using the 3′-side of the targeted locus using the primer set RArmF: 5′-CGCCCTATAGTGAGTCGTATTAC-3′ (SEQ ID NO:14) and ART2R: 5′-ATACATACGCGGCCGCGTCAATAAGTAAATACAAATAAAGTAATAAAAAATTATTG GC-3′ (SEQ ID NO:15). Correct targeting was determined by PCR using RArmF and ART2R15′-GTCACAGGTGACCAAAAAAAATTACTG-3′ (SEQ ID NO:16) primers. For the second round of targeting, PCR was performed again using the 3′-side of the targeted locus, however, the vector-specific primer was replaced with NeoF1: 5′-TTCTTGACGAGTTCTTCTGAGGGGATCAATTC-3′(SEQ ID NO:17). For the third round of targeting, a control PCR was performed for the 5′-side of the targeted locus using the primer set ART2F-1: 5′-GAGCCACCATGTCCAACTGGTTTAG-3′ (SEQ ID NO:18) and NeoR2: 5′-AAAGCGCCTCCCCTACCCGGTAGG-3′ (SEQ ID NO:19). Correct targeting was determined by using ART2EF: 5′-ACTGGGTCTAATGATGGCCACACGAC-3′ (SEQ ID NO:20). The null status was determined using a pair of Artemis exon 2 flanking primers that produce different sized products when amplified from an exon 2-containing allele or a Lox P site-containing allele. This PCR was performed using ART2 5′F: 5′-CCCTTGGGCTAAGGAATCCTCTGG-3′ (SEQ ID NO:21) and ART2 3′R: 5′-AATGTTTGCTTAAAAACACAAGTAGC-3′ (SEQ ID NO:22).

### Gene Targeting Strategy

In order to knock out the first allele of Artemis, the rAAV-Artemis exon 2 Neo virus was used. The relative targeting frequency was 3/176 or 1.7%. Once a correctly targeted clone was identified, the neomycin selection cassette was removed by Cre recombination (Ruis et al.). Briefly, the cells were transfected with the PML-Cre plasmid using Lipofectamine LTX after which they were plated at limited dilutions onto 10 cm dishes and allowed to form colonies. Approximately 2 weeks later, individual colonies were characterized for confirmation of the loss of one allele of Artemis exon 2 by PCR and for G418 sensitivity. The second round of targeting was methodology was identical to that used in the first round. 14 independent correctly gene targeted clones were produced from 1700 drug resistant clones (0.82% gene targeting frequency). Although at this time it was expected that some of these clones would by null for Artemis, PCR analysis using primers flanking exon 2 of Artemis, as well as an exon 2-specific primer, showed that Artemis in the HCT 116 cell line was at least triploid. This was perhaps not surprising since there is a large duplication on the q arm of one chromosome 10 (Masramon et al.); the same chromosome where the Artemis locus resides (Moshous et al.). After another round of Cre treatment, this time using CMV AdCre virus (Wang et al.), a third round of gene targeting was performed using rAAV-Artemis exon 2 Puro virus. Five correctly targeted clones were obtained out of 120 drug-resistant clones for a relative targeting frequency of 4.2%. Two of these clones (clone 15 and clone 18) were determined to be null for Artemis exon 2 based on PCR using exon 2 flanking primers ART2 5′F and ART2 3′R.

### Gene Targeting Efficiency In Artemis Null Cells

rAAV XRCC4 exon 4 Neo virus was used for viral infection as described above. G418 resistant single colonies (50) were isolated from 96-well plates and expanded to 24-well plates for isolation of genomic DNA. The harvested DNA was then subjected to PCR to determine correct targeting using the primer pair RArmF and XRCC4.4 ER2: 5′-GCCAAATAACACTAGATGTTAGGAAC-3′ (SEQ ID NO:23). To confirm the presence of the integrated vector the primer pair RArmF and XRCC4.4 RR: 5′-ATACATACGCGGCCGCGTCTATACAGAGCAATCACAATGG-3′ (SEQ ID NO:24) was used.

### Results

In order to determine if the loss of Artemis confers higher relative gene targeting frequencies, the HCT116 Artemis exon 2−/−/− (subclone 15.1) cells were used in an experiment in which XRCC4 exon 4 was targeted. Fifty drug-resistant clones that were also PCR-positive for rAAV were obtained. Seven of the 50 clones tested were determined to be correctly targeted; resulting in a relative gene targeting frequency of 14.0%. Gene targeting at this locus in the parental cell line was 22 correctly targeted clones from 2026 clones analyzed (compilation of three independent experiments) for a gene targeting frequency of 1.1%. Thus, the absence of Artemis resulted in a 12.7-fold (14.0% versus 1.1%) stimulation in the relative correct gene targeting frequency.

### Discussion

It was previously demonstrated that the frequency of rAAV-mediated gene targeting is higher in Ku70-reduced and DNA-PKcs-deficient human somatic cells. This may be due to the normal function of Ku (and probably DNA-PKcs) in suppressing other DNA double-strand break repair pathways, most notably homologous recombination (Fattah et al.). Here, this observation is extended to another C-NHEJ gene, Artemis. In Artemis-deficient human somatic cell lines, the frequency of relative rAAV-mediated gene targeting is improved by over an order of magnitude. Although the precise mechanism for this improvement is not unequivocally known, it is believed that it is via a different mechanism than that of Ku and/or DNA-PKcs. Specifically, it is it is believed that Artemis may be required to process the viral ITRs in order to permit random integration events (Inagaki et al.). Thus, in an Artemis-deficient cell line, the relative targeting frequency is increased because the total number of random integrations decreases. This feature has an extra attractive advantage for potential gene therapy studies with human patients, where random integration events must be kept to a minimum.

### BIBLIOGRAPHY

Carter B J (2004) Adeno-associated virus and the development of adeno-associated virus vectors: A historical perspective. Mol Ther 10:981-989.
Cattell, E., B. Sengerova, and P. J. McHugh. 2010. The SNM1/Pso2 family of ICL repair nucleases: from yeast to man. Environ Mol Mutagen 51:635-45.
Chen, I (2008) Finding the right target. Nature Struct. Mol. Biol. 15:699.
Fattah K R, Ruis B L, Hendrickson E A (2008) Mutations to Ku reveal differences in human somatic cell lines. DNA Repair 7:762-774.
Fattah F, Lichter N, Fattah K R, Oh S, and Hendrickson, E A (2008) Ku70, an essential gene, modulates the frequency of rAAV-mediated gene targeting in human somatic cells. Proc. Natl. Acad. Sci., USA 105:8703-8708.
Fattah F, Lee E H, Weisensel N, Wang Y, Lichter N, and Hendrickson E A (2010) Ku regulates the non-homologous end joining pathway choice of double-strand breaks in human somatic cells. PLoS Genetics, 6:e1000855.
Hastings P J, McGill C, Shafer B, Strathern J N (1993) Ends-in vs. ends-out recombination in yeast. Genetics 135:973-980.
Hendrickson E A, Huffman J L, Tainer J A (2006) in DNA Damage Recognition, Structural aspects of Ku and the DNA-dependent protein kinase complex, eds. Seide W, Kow Y W, Doetsch P (Taylor and Francis, New York), pp 629-684.
Hendrickson E A (2008) in Sourcebook of Models for Biomedical Research, Gene targeting in human somatic cells, ed. Conn P M (Humana, Totowa, N.J.), pp 509-525.
Heyer W D, Li X, Rolfsmeier M, Zhang X P (2006) Rad54: the Swiss Army knife of homologous recombination? Nucleic Acids Res 34:4115-4125.
Inagaki K, et al. (2007) DNA palindromes with a modest arm length of greater, similar 20 base pairs are a significant target for recombinant adeno-associated virus vector integration in the liver, muscles, and heart in mice. J Virol 81:11290-11303.
Inagaki, K., C. Ma, T. A. Storm, M. A. Kay, and H. Nakai. 2007. The role of DNA-PKcs and artemis in opening viral DNA hairpin termini in various tissues in mice. J Virol 81:11304-21.
Khan, I. F., R. K. Hirata, and D. W. Russell. 2011. AAV-mediated gene targeting methods for human cells. Nat Protoc 6:482-501.
Kohli, M., C. Rago, C. Lengauer, K. W. Kinzler, and B. Vogelstein. 2004. Facile methods for generating human somatic cell gene knockouts using recombinant adeno-associated viruses. Nucleic Acids Res 32:e3.
Kurosawa, A., and N. Adachi. 2010. Functions and regulation of Artemis: a goddess in the maintenance of genome integrity. J Radiat Res (Tokyo) 51:503-9.

Li G, Nelsen C, Hendrickson EA (2002) Ku86 is essential in human somatic cells. Proc Natl Acad Sci USA 99:832-837.
Ma, Y., U. Pannicke, K. Schwarz, and M. R. Lieber. 2002. Hairpin opening and overhang processing by an Artemis/DNA-dependent protein kinase complex in nonhomologous end joining and V(D)J recombination. Cell 108:781-94.
Masramon, L., M. Ribas, P. Cifuentes, R. Arribas, F. Garcia, J. Egozcue, M. A. Peinado, and R. Miro. 2000. Cytogenetic characterization of two colon cell lines by using conventional G-banding, comparative genomic hybridization, and whole chromosome painting. Cancer Genet Cytogenet 121:17-21.
Miller, D. G., G. D. Trobridge, L. M. Petek, M. A. Jacobs, R. Kaul, and D. W. Russell. 2005. Large-scale analysis of adeno-associated virus vector integration sites in normal human cells. J Virol 79:11434-42.
Moshous, D., I. Callebaut, R. de Chasseval, B. Corneo, M. Cavazzana-Calvo, F. Le Deist, I. Tezcan, O, Sanal, Y. Bertrand, N. Philippe, A. Fischer, and J. P. de Villartay. 2001. Artemis, a novel DNA double-strand break repair/V(D)J recombination protein, is mutated in human severe combined immune deficiency. Cell 105:177-86.

Nicolas, N., D. Moshous, M. Cavazzana-Calvo, D. Papadopoulo, R. de Chasseval, F. Le Deist, A. Fischer, and J. P. de Villartay. 1998. A human severe combined immunodeficiency (SCID) condition with increased sensitivity to ionizing radiations and impaired V(D)J rearrangements defines a new DNA recombination/repair deficiency. J Exp Med 188:627-34.
Rooney, S., J. Sekiguchi, C. Zhu, H. L. Cheng, J. Manis, S. Whitlow, J. DeVido, D. Foy, J. Chaudhuri, D. Lombard, and F. W. Alt. 2002. Leaky Scid phenotype associated with defective V(D)J coding end processing in Artemis-deficient mice. Mol Cell 10:1379-90.

Ruis B, Fattah KR, and Hendrickson EA (2008) DNA-PK_{cs} regulates proliferation, telomere length and genomic stability in human somatic cells. Mol. Cell. Biol. 28:6182-6195.
Russell D W, Hirata R K (1998) Human gene targeting by viral vectors. Nat Genet. 18:325-330.

Spagnolo L, Rivera-Calzada A, Pearl L H, Llorca 0 (2006) Three-dimensional structure of the human DNA-PKcs/Ku70/Ku80 complex assembled on DNA and its implications for DNA DSB repair. Mol Cell 22:511-519.
Thomas K R, Capecchi M R (1987) Site-directed mutagenesis by gene targeting in mouse embryo-derived stem cells. Cell 51:503-512.
van Veelen L, Wesoly J, Kanaar R (2006) in DNA Damage Recognition, Biochemical and cellular aspects of homologous recombination, eds Seide W, Kow Y W, Doetsch P (Taylor and Francis, New York), pp 581-607.
Wang Y, Ghosh G, and Hendrickson E A (2009) Ku86 regulates lethal telomere rapid loss in somatic human cells. Proc. Natl. Acad. Sci., USA, 106:1243-12435.
Yan, Y., S. Akhter, X. Zhang, and R. Legerski. 2010. The multifunctional SNM1 gene family: not just nucleases. Future Oncol 6:1015-29.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to increase gene targeting frequency comprising inhibiting expression of at least one gene of a DNA double strand break (DSB) repair pathway or by inhibiting activity of at least one protein of a DNA DSB repair pathway so as to provide increased gene targeting frequency as compared to a cell in which expression and/or activity has not been inhibited.

2. A method to reduce stable random exogenous DNA integration comprising inhibiting expression of at least one gene of a DNA DSB repair pathway or by inhibiting activity of at least one protein of a DNA DSB repair pathway so as to provide decreased random DNA integration as compared to a cell in which expression and/or activity has not been inhibited.

3. A method to increase stable targeted DNA integration comprising inhibiting expression of at least one gene of a DNA DSB repair pathway or by inhibiting activity of at least one protein of a DNA DSB repair pathway so as to provide increased targeted DNA integration as compared to a cell in which expression and/or activity has not been inhibited.

4. The method of claim 3, wherein the random DNA integration is viral DNA integration.

5. The method of claim 1, wherein the DNA DSB repair pathway is the C-NHEJ pathway.

6. The method of claim 1, wherein the DNA DSB repair pathway is the A-NHEJ pathway.

7. The method of claim 1, wherein the gene is selected from the group consisting of Ku70, Ku86, DNA-PK_{cs}, Artemis, LIGIV, XLF, XRCC4 or a combination thereof.

8. The method of claim 1, wherein the gene is selected from the group consisting of Artemis, LIGIV, XLF, XRCC4 or a combination thereof.

9. The method of claim 1, wherein the gene is selected from the group consisting of LIGIII, PARP1, RAD54B, XRCC1, XRCC3, MRE11, NBS1, RAD50, CtIP, FEN1, EXO1, BLM or a combination thereof.

10. The method of claim 1, wherein expression is transiently inhibited.

11. The method of claim 1, wherein expression permanently inhibited.

12. The method of claim 1, wherein the protein is selected from the group consisting of Ku70, Ku86, DNA-PK_{cs}, Artemis, LIGIV, XLF, XRCC4 or a combination thereof.

13. The method of claim 12, wherein the protein is selected from the group consisting of Artemis, LIGIV, XLF, XRCC4, or a combination thereof.

14. The method of claim 1, wherein the protein is selected from the group consisting of LIGIII, PARP1, RAD54B, XRCC1, XRCC3, MRE11, NBS1, RAD50, CtIP, FEN1, EXO1, BLM, or a combination thereof.

15. The method of claim 1, wherein the protein is inhibited by a small molecule or expression of the protein is inhibited by antisense, siRNA or shRNA.

16. The method of claim 15, wherein the small molecule is an inhibitor of a lipid-modifying enzyme, such as a kinase inhibitor.

17. The method of claim 15, wherein the small molecule is NU7441, wortmannin, NU7026, vanillin, LY 294002, PX866 or a combination thereof.

18. The method of claim 15, wherein DNA-PK_{cs} is inhibited by a small molecule inhibitor, wherein the small molecule inhibitor is an inhibitor of a lipid-modifying enzyme, such as a kinase inhibitor.

19. The method of claim 18, wherein the small molecule inhibitor is selected from the group consisting of NU7441, wortmannin, NU7026, vanillin, LY 294002, PX866 or a combination thereof.

20. The method of claim 15, wherein DNA-PK_{cs} is inhibited by a small molecule inhibitor selected from the group consisting of wortmannin, NU7026, vanillin, LY 294002, PX866 or a combination thereof.

21. The method of claim 1, wherein the telomeres are not dysfunctional.

22. The method of claim 1, wherein the gene integration and/or targeting is mediated by a retrovirus, rAAV, dsDNA, ssDNA, zinc finger nuclease, homing nuclease, meganuclease, transcription activator like (TAL) effector nuclease or a combination thereof.
